(19) United States
(12) Patent Application Publication
(10) Pub. No.: US 2013/0018062 A1
(43) Pub. Date: Jan. 17, 2013

(54) Pain therapy with metabolism blocked antifolates

(71) Applicant: Madhavan G. Nair, Mobile, AL (US)

(72) Inventor: Madhavan G. Nair, Mobile, AL (US)

(21) Appl. No.: 13/135,668

(22) Filed: Jul. 12, 2011

(51) Int. Cl. A61K 31/517; A61P 29/00

(52) U.S. Cl. 514/266.24; 514/266.2; 514/266.4

(57) ABSTRACT

The perception of pain precedes the manifestation of measurable and quantifiable clinical symptoms of most diseases. Pain is an early warning signal at the beginning of a physical abnormality. The present invention discloses novel methods to ameliorate or reverse diseases at their warning stages by therapeutic intervention of pain before a definite clinical diagnosis of the abnormality can be made. Unprecedented methods to eliminate pain caused by inflammatory autoimmune response or inflammatory allergic response or malignant and benign neoplastic growth by administering metabolism blocked antifolate compounds are described.

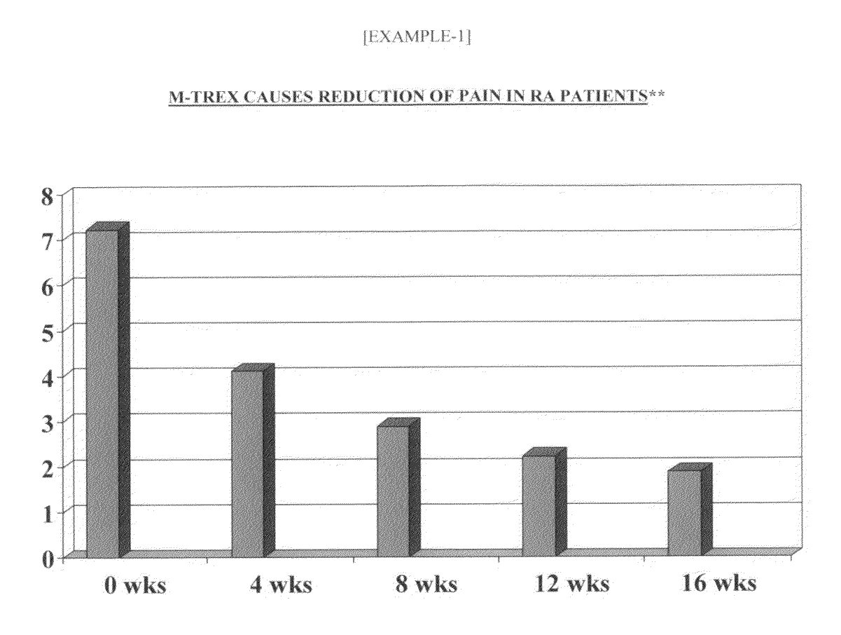

### FIELD OF INVENTION

This invention encompasses the use of metabolism blocked Antifolates as novel therapies to reduce or ameliorate pain caused by a variety of abnormal physical abnormalities.

### BACKGROUND OF THE INVENTION

Physical abnormalities that cause pain include but are not limited to neoplasms, inflammation caused by autoimmune responses, inflammation caused by allergic responses, cardiovascular inflammation, ischemia, abnormal tissue growth and proliferation, tissue destruction and arterial stenosis. Some specific causes of pain are cancer, heart disease, cardiovascular disease, peripheral artery disease, rheumatoid arthritis, juvenile rheumatoid arthritis, psoriasis, inflammatory bowel disease and uvetis.

Folic acid is a vitamin that is needed for normal tissue growth and proliferation. Folic acid is converted to more than 150 metabolites in the human and each of them separately or collectively mediates a specific biological function. [M. G. Nair. “ANTIFOLATES”. In “Cancer Growth and Progression—Cancer Control in Man”. Chapter 10. H. E. Kaiser (Ed). Kluwer Academic Publishers (1990). M. G. Nair. “Chemistry of Antifolates”. In “The Chemistry of Antitumor Agents”. D. E. V. Wilman (Ed) Blackie and Sons (Lond); Chapman and Hall (USA) Chapter—7 (1990)].

Antifolates are compounds that interfere with the complex folate metabolism at various stages and they interfere with cell growth and proliferation. [M. G. Nair. Antifolate drugs in Chemotherapy (review). Drug Discovery Today 4: 492-494 (1999).]

Antifolates are further classified as either classical or non-classical. Classical Antifolates such as methotrexate normally retain a glutamate moiety as part of the chemical structure and they undergo a process known as “polyglutamylation” first discovered and documented by C. M. Baugh, C. L. Krumdieck and M. G. Nair. [C. M. Baugh, C. L. Krumdieck and M. G. Nair. Polygammaglutamyl metabolites of methotrexate. Biochem. Biophys. Res. Comm. 52:27 (1973)] and Nair and Baugh in 1973. [M. G. Nair and C. M. Baugh. The synthesis and biological evaluation of Polygammaglutamyl derivatives of methotrexate. Biochem. 12:3923 (1973).]

Polyglutamylation of Antifolates is a metabolic process that results in the elaboration of Antifolates to more complex molecules that retain within the cells for prolonged periods. While polyglutamylation exacerbates Antifolate potency they are also very toxic due to a number of reasons. Thus polyglutamylation of Antifolates is a process of activation of the drug but at the expense of undesirable toxicity. [A. Amato, M. Fayard, J. Lariccia, J. Mallet, S. Miles and M. G. Nair. Metabolism-based Antifolate drug design: MDAM and M-Trex. Pharmacology and therapeutics, New Millennium. Ed. S. K. Gupta. Pp. 204-212 (2001).]

Methotrexate and its congeners belonging to the same generic species defined as “classical” also undergo another metabolic process in humans referred to as 7-hydroxylation. This is mainly a deactivation process whereby the drug looses its Antifolate activity by virtue of loss of target enzyme inhibition. The hydroxylated product is not only inactive as an inhibitor of target enzyme but also capable of further metabolism to polyglutamates that exert very serious toxicity to the host. [A. Abraham, R. Pati, J. H. Galivan, M. S. Rhee, J. J. McGuire, R. L. Kisliuk, F. H. Hausheer and M. G. Nair. Aldehyde oxidase mediated 7-hydroxylation of antifolates and its therapeutic relevance. Cellular Pharmacology. 3; 29-34 (1996); [S. Cao, A. Abraham, M. G. Nair, R. Pati, J. H. Galivan, F. H. Hausheer and Y. M. Rustum. Polyglutamylation of the dihydrofolate reductase inhibitor (γ-methylene-10-deazaaminopterin is not essential for antitumor activity. Clinical Cancer Research. 2; 707-712 (1996)].

The biochemical pharmacology of Antifolate polyglutamates, their hydroxylated products and metabolites were studied exhaustively by M. G. Nair and his collaborators and from these studies it became apparent that both polyglutamylation and 7-hydroxylation of Antifolates are undesirable processes that manifest in unacceptable host toxicity. In early 1990s Nair coined in the term; “non-polyglutamatable antifolates” to describe several of his new Antifolate compounds that could not be elaborated to polyglutamates, γ-methylene-10-deazaaminopterin [MDAM] being the model compound that underwent a series of human testing as a therapy to treat human cancers. [M. G. Nair. Two New Non-polyglutamatable Antifolates. U.S. Pat. No. 5,073,554 (1991)]. Other examples of this series were MEDAM, 3-Hydroxy-10-DAM and 3-Hydroxy-EDAM. [M. G. Nair and Ann Abraham. Three New Non-polyglutamatable 10-Deazaaminopterins. U.S. Pat. No. 4,996,207 (1991)]

Being encouraged by the clinical results obtained with MDAM, Nair later developed classical Antifolates that could neither be elaborated to polyglutamates nor hydroxylated compounds. [M. G. Nair. Metabolically inert anti-inflammatory and anti-tumor antifolates. U.S. Pat. No. 5,912,251 (1999)]. To describe these compounds Nair introduced the term “metabolism blocked” in Antifolate terminology. The representative example of such a “Metabolism Blocked” Antifolate compound developed by Nair is known as M-Trex as the racemic mixture. These two enantiomers of M-Trex are now commercially known as CH-1504 and CH-4051 respectively. [M. G. Nair, M. L. Fayard, J. M. Lariccia, A. E. Amato, J. J. McGuire, J. H. Galivan and R. L. kisliuk. Metabolism-blocked folate analog inhibitors of dihydrofolate reductase-1: Synthesis and biological evaluation of Mobiletrex. Med. Chem. Research. 9:176-185, 1999.]

Nair and Kisliuk also separated the L-enantiomer of M-Trex by chiral chromatography and evaluated its biological activity. [M. G. Nair and R. L. kisliuk. Metabolism-blocked Antifolates: 3. Enantiomers of 4′-methylene-5, 8-10-trideazaaminopterin (M-Trex). Proc. Amer. Assoc. Cancer. Res. 42: 294 (1583) 2001].

### PREFERRED EMBODIMENTS

None of the metabolism blocked Antifolate compounds, including the representative compounds developed by Nair known as M-Trex (MobileTrex) or L-M-Trex is currently approved for use to treat any form of human disease, except on an experimental basis. Specifically, classical Metabolism Blocked Antifolates are not currently approved as therapies for any disease that causes Pain. Some of the representative inflammatory diseases (including some caused by auto immune response, allergic response, tissue damage and abnormal tissue proliferation) that causes the onset of pain and discomfort before a full measurable diagnosis is made include: Coronary Heart Disease (CHD), Cardiomyopathy, Heart Failure, Coronary Artery Disease (CAD), Ischemia, Angina Pectoris, Peripheral Artery Disease (PAD); Asthma, Chronic Obstructive Pulmonary Disease (COPD); Rheumatoid Arthritis, Juvenile Rheumatoid Arthritis; Psoriasis, Uvetis, (itching and burning), Oesteo-arthritis; Inflammatory Bowel Disease (IBD), Chron's Disease (visceral pain) and dozens of benign or malignant neoplastic diseases. The data presented in this section (see below) establish that Metabolism Blocked Antifolates (Claim-1) that encompass this new invention can significantly reduce pain due to inflammatory diseases caused by auto immune response (RA) or allergic response (Asthma or COPD); can ameliorate or shrink neoplasms that causes pain (cancers) and can restore normal lung function of rabbits challenged with allergens.

The biochemical and pharmacological studies of both M-Trex and L-M-Trex that were formulated as their respective Sodium Salts or Ammonium Salts by Nair et al. [U.S. Pat. No. 5,912,251] established that they possess superior antifolate activity relative to metabolizable (that undergo polyglutamylation and hydroxylation) antifolates such as the well know antifolate drug methotrexate or 10-Deazaaminopterin [G. Alarcon, O. Castaneda, W. J. Koopman, C. L. Krumdieck, and M. G. Nair. 10-Deazaaminopterin: A New Arthritis Remittive Drug. U.S. Pat. No. 5,030,634 (1991)].

An efficacy study of 10-Deazaaminopterin (10-DAAM) versus methotrexate in patients with rheumatoid arthritis established that 10-DAAM is very effective in reducing Pain and in fact superior to the efficacy of methotrexate. [G. S. Alarcon, M. O. Castaneda, M. G. Nair, A. Berrocal, E. Paz, W. J. Koopman, and C. L. Krumdieck. Controlled trial of methotrexate versus 10-deazaaminopterin in the treatment of rheumatoid arthritis. Annals. Rheumatic Diseases, 51, 600 (1992)].

Of special importance was the novel observation of the ability of M-Trex to substantially reduce Pain in human subjects who were suffering from inflammation caused by autoimmune response. [O. Castaneda and M. G. Nair. Controlled trial of methotrexate versus CH-1504 in the treatment of Rheumatoid Arthritis. The J. Rheumatology. 33, 862-64, 2006.] In tissue culture models M-Trex exhibited outstanding activity in inhibiting abnormal proliferation of a number of human cells. Another unique observation with M-Trex was its ability to shrink human tumor xenografts in immuno-compromised mice. The ability of metabolism blocked antifolate M-Trex to shrink rapidly proliferating human tissues is representative and results in Pain reduction secondary to such proliferation. [EXAMPLE-1]** ** Pain assessment was made using the VAS scale every four weeks. Patients were treated with a daily oral dose of 7.5 mg/day of M-Trex during the duration of measurements. The laboratory values of these patients established that the pain reduction was not associated with any measurable toxicity. Pain as assessed by VAS was reduced from a scale of 7.2 to 1.8.

The pain reduction in humans were also accompanied by amelioration of inflammation caused by autoimmune response; the ideal model used for the study being rheumatoid arthritis (RA). M-Trex was also very effective in reducing inflammatory responses in animals challenged with allergens. [Madhavan G. Nair, U.S. Pat. No. 5,912,251] From this novel observation of the ability of Metabolism Blocked Antifolates to reduce pain and shrink tissue growth caused by inflammatory events secondary to autoimmune response or allergic response has led to the rationalization of this new and novel invention namely “Pain Therapy with Metabolism Blocked Antifolates’.

M-TREX LABORATORY VALUES.^{@}

| | VISIT 2 | VISIT 3 | VISIT 4 | VISIT 5 | VISIT 6 | VISIT 7 |
|---|---|---|---|---|---|---|
| HEMOGLOB | 11.77 | 12.43 | 12.18 | 12.26 | 12.56 | 12.48 |
| GLUCOSE | 83.8 | 95 | 87.5 | 88.1 | 92.4 | 90.3 |
| CREATININE | 0.79 | 0.95 | 0.85 | 0.85 | 0.92 | 0.84 |
| SGOT | 14.6 | 16 | 16 | 17.3 | 14.9 | 15.2 |
| SGTP | 17.75 | 17 | 25 | 20 | 18.21 | 18 |
| ALK. | 107.75 | 86 | 89.3 | 87.1 | 85.42 | 86.5 |
| PHOSPHAT | | | | | | |

^{@}A daily dose of M-Trex at 7.5/mg per day was administered to patients with Rheumatoid Arthritis. The laboratory values were determined on each visit. At this dose that was very effective in reducing pain and inflammation (swollen joints) the laboratory values did not indicate any toxicity.

### Chemical Structure of M-Trex*

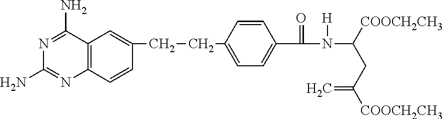

*Chemical structure of M-Trex diethyl ester is depicted above. In M-Trex the two ethyl groups [CH₂CH₃] are replaced with two hydrogen atoms [—H]

The perception of pain precedes the manifestation of any clinically measurable patho-physiological symptoms in almost all cases with very few exceptions. There fore it is extremely important to treat pain before any underlying abnormalities are physically uncovered by common diagnostic techniques. Since pain is a warning sign of underlying abnormalities, therapeutic intervention of pain in the earliest stage would tend to ameliorate the underlying disease even before it is uncovered. As an example, if Ischemic pain can be treated with a drug successfully it becomes apparent that the conditions causing Ischemia can be eliminated by therapy, even before the patho-physiological symptoms causing the Ischemia could be uncovered by radiological or other techniques weeks or months after the onset of such pain. The onset and persistence of pain due to Angina before it becomes severe as an emergency can last for several months or years. Thus treating angina pain at the onset can ameliorate the underlying causes before surgical or therapeutic interventions become mandatory after the disease has progressed to an emergency state. Thus Pain Treatment at the onset can also be successful as a prophylactic procedure to prevent progression, reverse the course or eradication of the underlying disease.

Coronary Heart Disease (CHD), Cardio-vascular diseases (CVD) and Stroke are of the same generic species by virtue of Inflammation being the common underlying cause of their occurrence. In fact an article by the American Heart Association entitled “Inflammation, Heart Disease and Stroke. The role of C-Reactive Protein” stressed that markers of Inflammation are more accurate and reliable predictive factors than total cholesterol levels in humans. [www.americanheart.org/presenter.jhtml?presenter=4648]. The most accurate predictive factor for the incidence of CHD, CVD and stroke is elevated levels of C-Reactive Protein (CRP). In a clinical study conducted by Castaneda and Nair as early as 2002 it was observed that arthritic patients taking metabolism blocked antifolate compound M-Trex showed significant and consistent reduction of their CRP levels.

This observation led Nair to promulgate a theory that Metabolism blocked Antifolates, specifically M-Trex would be useful as therapies for CHD, CVD and Stroke and thereby ameliorates also the discomfort and Pain associated with them. About eleven million Americans suffer from silent Ischemia and angina pectoris combined; the diseases that manifest dull, diffused and severe pain depending on the severity of ischemic condition. Early therapeutic intervention of ischemic pain caused by inflammation of arteries may in fact slow the progression, reverse or completely abrogate the disease. At the earliest sign of ischemic Pain, therapeutic intervention with metabolism-blocked antifolates might result in at least stable disease at worst, or reverse or ameliorate the inflammation. The need to perform diagnosis using echocardiography, angiography, CT scan, Doppler ultrasonography, MRI or PET and accompanying surgical or therapeutic interventions can be avoided at a later stage of advanced level of disease, if pain therapy is instituted at the onset of Ischemia with metabolism blocked antifolates.

### Example-2

Plasma CRP Levels at Visits 4, 8, 12 and 16 Weeks of Patients on
Therapy with M-Trex for Rheumatoid Arthritis.##

| | | | | |
|---|---|---|---|---|
| CRP | 22.1 | 20.7 | 17.7 | 13.6 |

##Data taken from clinical trial results: [O. Castaneda and M. G. Nair. Controlled trial of methotrexate versus CH-1504 in the treatment of Rheumatoid Arthritis. The J. Rheumatology. 33, 862-64, 2006.]

### Example-3

M-TREX IS A SUPERIOR ANTIINFLAMMATORY AGENT
IN A RABBIT MODEL OF HUMAN ASTHMA@@
Comparative Effects of MTX and M-Trex on Allergen Induced
Asthmatic Responses and Bronchial Hyper-Responsiveness
(BHR) in Allergic Rabbits.

Asthmatic
Response

| Compound | Early | Late | BHR |
|---|---|---|---|
| MTX | 29 | 37 | 32 |
| M-Trex | 52 | 58 | 47 |
| Theophylline | 45 | 49 | 39 |

@@Data were taken from M. G. Nair, U.S. Pat. No. 5,912,251. The above data showing the superiority of Metabolism Blocked Antifolate M-Trex over methotrexate or the drug of choice Theophylline in alleviating inflammation caused by allergic response illustrates the utility of M-Trex as claimed in this Invention.

There are three general accepted methods for the evaluation of Pain in patients suffering from a number of diseases. They are Visual Analog Scale (VAS); Numeric rating Scale (NRS) and Verbal Categorical Rating Scale (NRS). Of these three assessment methods VAS is considered to be most reliable in a majority of cases to evaluate and quantify the magnitude of the perception of Pain. Clinical trials conducted by Castaneda and Nair used VAS to assess pain intensity in RA patients receiving 10-DAAM in one trial [G. Alarcon, O. Castaneda, W. J. Koopman, C. L. Krumdieck, and M. G. Nair. U.S. Pat. No. 5,030,634 (1991)] and M-Trex (CH-1504) in another trial conducted separately. Almost all cases of cancer Pain is due to tissue inflammation, tissue compression and tissue damage secondary to rapid proliferation of cells. Therefore any therapeutic intervention that shrinks proliferating tissues can bring about significant reduction in Pain. Shown below is the efficacy of M-Trex in shrinking human colon cancer transplanted and growing in nude mice.

### Example-4

Antitumor Activity and Toxicity** of MTX and M-Trex by i.v. Push
Daily × 5 in Nude Mice Bearing Human HCT-8 Colon Xenograft.

| Drug¹ (mg/kg/day) | MTGI (%)² | TD (days)³ | T/C % |
|---|---|---|---|
| Control | — | 2.6 ± 0.8 | — |
| M-Trex (4.0 mg/kg/day) | 91.4 ± 2.2 | 16.2 ± 3.2 | 623.08 |
| MTX (1.0 mg/kg/day)³³ | 30.0 ± 9.0 | 4.6 ± 0.6 | 176.92 |

¹The doses used are the Maximum Tolerated doses for each drug on this schedule
²Maximum Tumor Growth Inhibition (% of control)
³Tumor Doubling Time
**None of the animals had weight loss above 20% on this schedule. Animals on MTX, M-Trex and MDAM experienced weight loss of approximately 14, 12 and 8 percent respectively. All animals regained weight to original levels by day 15.

Cell growth regression as a means of alleviating Pain especially cancer pain is further illustrated by the remarkable ability of M-Trex to inhibit the growth of a number of human cancer cells in culture. If the tested cancers were growing in a cancer patient the data provided below is illustrative of the ability of M-Trex to significantly reduce pain by decompression of normal tissues by tumor shrinkage and tumor regression.

### Example-5

INHIBTION OF TUMOR GROWTH BY M-REX AND MTX
Growth Inhibition (GI₅₀ & TGI) of Human Tumor Cells
by M-Trex and MTX**

| | |
|---|---|
| Log₁₀ GI₅₀ | TGI |

| Cell Line | M-Trex | M-Trex | MTX |
|---|---|---|---|
| Leukemia | | | |
| CCRF-CEM | <−8.00 | <−8.00 | −3.77 |
| MOLT-4 | <−8.00 | −7.40 | −3.55 |
| RPMI 8226 | <−8.00 | −7.44 | −3.66 |
| SR | <−8.00 | <−8.00 | −3.50 |
| K562 | <−8.00 | <−8.00 | −3.30 |
| Breast Cancer | | | |
| MCF-7 | <−8.00 | −6.32 | — |
| MCF7-ADR-RES | <−8.00 | −7.26 | — |
| CNS Cancer | | | |
| SF 539 | <−8.00 | −6.3 | −3.66 |

The preceding data collected at the National Cancer Institute summarize the ability of M-Trex to inhibit 50% of the growth of selected tumor cells in culture. The values are expressed as Log₁₀ GI₅₀ values all of which are lower than 10⁻⁸M. In the same table a comparison is made of the respective values of TGI (Total Growth Inhibition) for M-Trex and MTX. The data show that M-Trex is approximately 1,000 to 10,000 times more effective than MTX to cause total growth inhibition of these human tumor cell lines. In addition, the Log₁₀ GI₅₀ values of M-Trex was lower than −8.00 for the following cell lines: NSCLC, A549/ATCC and NCI H23; Colon Cancer, HCT 116, HCT 29 and SW 620; CNS Cancer, SF 268; Melanoma M 14 and UACC 62; Ovarian Cancer, OVCAR-8; Renal Cancer, ACHN, CAKI-1, SN 12C, and UO 31; Prostate Cancer, PC 3 and Breast cancer, MDA-MB-435 and MDA-N. The MG_MID value of MTX for all 49 human tumor cell lines evaluated at NCI-DTP program was −7.05. This strikingly superior activity of M-Trex must have its origin in the metabolic blocks and the resultant constant steady state levels of the drug on continuous exposure. The tumors and sarcomas are solid while leukemia and lymphoma are liquid tumors.

### Example-6

Reduction of Morning Stiffness and Swollen Joints in patients
with Rheumatoid Arthritis Treated with M-Trex.

| MS | 99′ | 26′ | 22′ | 20′ | 14′ |
|---|---|---|---|---|---|
| S. Joints | 12.5 | 5.6 | 3.5 | 3.0 | 2.9 |

MS (Morning Stiffness); S. Joints (Swollen Joints) were remarkably responsive to M-Trex therapy. Stiffness exacerbates Pain especially in joints by virtue of inflexibility and reduction is blood circulation. Swelling of joints is secondary to inflammation caused by autoimmune response in patients with Rheumatoid Arthritis. The clinical study results summarized (Castaneda and Nair) in Example-6 show that both the severity of Morning Stiffness and the number of Swollen Joints were dramatically reduced by the administration of metabolism blocked antifolate M-Trex. The initial numbers represents the values at the start of the study before the treatment and the remaining four numbers are values determined during each visit while taking M-Trex medication.

In joints stiffness (duration measured in minutes as above) is caused by inflammation of the synovium that lines the joint. Inflammation due to bone spurs as in Oesteo-arthritis causes Pain. Thus reduction of stiffness reduces the severity of inflammation and Pain. Abnormal tissue growth characterized by benign and malignant neoplasms cause stiffness due to compression of tissues, interfering with blood flow and irritation of the autonomic nervous system. Metabolism blocked antifolates are shown to be capable of decompression of tissues by interfering with tissue growth as shown in Examples 4, 5 and 6. These examples establish that metabolism blocked antifolates can decompress cancer tissues and ameliorate cancer pain.

Examples 1-6 provided herein are clearly supportive of the hypothesis and illustrate the utility and practice of this Invention of ameliorating Pain using metabolism blocked antifolates or any of their pharmaceutically acceptable salts such as sodium salt, potassium salt, calcium salt, magnesium salt or organic salts represented by acetate, trifluoro acetate, citrate, tartrate, ammonium salt, triethylammonium salt, or other salts such as hydrochloride or hydrobromide.

### An Unmet Need and its Significance:

Antifolates are neither used to treat cardiovascular diseases (CVD) nor pain arising from CVD. This invention therefore has dual significance; that is treatment of an underlying disease causing pain and the Pain itself for which no antifolate therapy is currently available. Likewise inflammatory diseases resulting from autoimmune responses, inflammatory diseases caused by allergic responses or abnormal tissue growth and proliferation characterized by various benign and malignant neoplastic diseases exhibit intrinsic or acquired resistant to the gold standard antifolate treatment with methotrexate in about 30-45% of patients with no reduction in Pain. This resistance to methotrexate treatment is attributed to its metabolic deactivation in certain patient populations with higher amounts of the deactivating enzyme Aldehyde Oxidase. [Nair, et al. Cellular Pharmacology. 3; 29-34 (1996] Aldehyde Oxidase a liver based enzyme that has broad specificity in oxidizing various substrates. Aldehyde Oxidase deactivates metabolizable antifolates including methotrexate and 10-DAAM to the corresponding hydroxy derivatives. The resulting hydroxy derivatives have no therapeutic potency per se but they are also subject to further metabolism to compounds that exhibit undesirable toxicity.

The metabolism Blocked antifolate compounds of this Invention are not substrates of Aldehyde Oxidase and they are not susceptible to oxidative deactivation. Thus the methotrexate resistant patient population will respond to treatment by metabolism blocked antifolates of this Invention resulting in dramatic reduction of Pain that otherwise would have been impossible. This Invention uniquely addresses an unmet need to reduce pain in patients with inflammatory diseases who are refractive or resistant to conventional therapies. The reduction or amelioration of Pain associated with CHD, CVD, CAD, PAD, Angina, Ischemia and arterial stenosis that affect in excess of 25 million people in United States alone per year is achievable with the unique methods provided in this Invention. Additionally, there are several dozens of inflammatory diseases that affect human beings throughout the world causing very serious to moderate pain and discomfort. The significant reduction or elimination of pain and discomfort in patients with rheumatoid arthritis (caused by autoimmune response) and in animal models of human Asthma (caused by allergic response) has been established conclusively with the use of metabolism blocked antifolate compound M-Trex, in the preceding five examples. EXAMPLE-1 Demonstrates reduction of Pain in patients treated with metabolism blocked antifolate M-Trex. EXAMPLE-2. Shows reduction of CRP levels, that is one of the most reliable markers of inflammation (especially ischemia, CVD, CAD, CHD, PAD, arterial stenosis etc.) and Pain in patients treated with M-Trex. EXAMPLE-3. Shows restoration of lung function by M-Trex in Rabbits challenged with allergen (Pain and discomfort due to inflammatory allergic response). EXAMPLE-4. Demonstrates decompression and shrinkage of abnormal tissue growth causing Pain. EXAMPLE-5. Shows inhibition of growth of human tumors that causes pain by compression of normal tissues (cancer Pain). EXAMPLE-6. Demonstrates amelioration of Stiffness and Swelling two main causes of Pain in patients treated with M-Trex.

Based on the above disclosures, I claim the following as inventive and patentable embodiments:

1. A method to treat pain arising from inflammatory autoimmune response or inflammatory allergic response or neoplastic growth by administering a metabolism blocked antifolate compound of the following chemical structure in an amount that is therapeutically effective and nontoxic to ameliorate the said pain in a human being or warm-blooded animal. 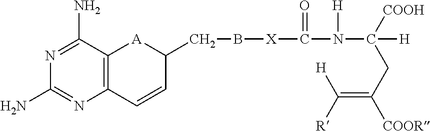
A is selected from a group consisting of: ═C—H; ═C—CH3; ═C—C2H5
B is selected from a group consisting of the following:
—CH₂—; —CH₂—CH₂—; —C(CH₃)—H; —C(C₂H₅)—H; —NH—; —NCH₃—; —O—; —S—
R′ is selected from —H; —CH3; —C2H5
R″=—H or —CH3
X is selected from the group consisting of the following moieties: 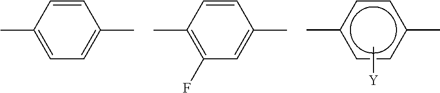
Where, Y is selected from a group consisting of —F, —Cl, —CH₃, —C₂H₅ placed at any of the four positions of the benzene ring by substitution of —H atoms, as mono, di, tri, or tetra substitution. Or one of the following moieties: 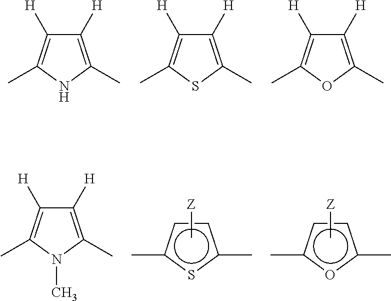
Where, Z is selected from a group consisting of —F, —Cl, —CH₃, or —C₂H₅ placed at any of the two positions of the hetero-cyclic ring by mono substitution or di substitution by replacing one or two hydrogen atoms respectively.

2. A method to treat pain arising from inflammatory autoimmune response or inflammatory allergic response or neoplastic growth by administering a metabolism blocked antifolate compound of claim-1 with or without a pharmaceutically acceptable carrier or diluent in an amount that is therapeutically effective and nontoxic to ameliorate the said pain in a human being or a warm-blooded animal.

3. A method to treat pain arising from inflammatory autoimmune response or inflammatory allergic response or neoplastic growth by administering a pharmaceutically acceptable salt of a metabolism blocked antifolate compound of claim-1 with or without a pharmaceutically acceptable carrier or diluent in an amount that is therapeutically effective and nontoxic to ameliorate the said pain in a human being or a warm-blooded animal.

4. A method to treat pain arising from inflammatory autoimmune response or inflammatory allergic response or neoplastic growth by administering the salt of a metabolism blocked antifolate compound according to claim-3 where the said salt is selected from the group: sodium, potassium, calcium, magnesium, ammonium, dimethylammonium, trimethylammonium, trifluoroacetate, acetate, citrate or tartrate.

5. A method to treat pain arising from rheumatoid arthritis or Juvenile rheumatoid arthritis by administering the salt of a metabolism blocked antifolate compound according to claim-1 with or without a pharmaceutically acceptable carrier or diluent in an amount that is therapeutically effective and nontoxic to ameliorate the said pain in a human being or a warm-blooded animal.

6. A method to treat pain arising from rheumatoid arthritis or Juvenile rheumatoid arthritis by administering a metabolism blocked antifolate compound according to claim-3 with or without a pharmaceutically acceptable carrier or diluent in an amount that is therapeutically effective and nontoxic to ameliorate the said pain in a human being or a warm-blooded animal.

7. The method to treat pain according to claim-5 where the pain arises from psoriasis or psoriatic arthritis.

8. The method to treat pain according to claim-6 where the pain arises from psoriasis or psoriatic arthritis.

9. The method to treat pain according to claim-7 where the pain arises from inflammatory bowel disease or Crohn's disease.

10. The method to treat pain according to claim-8 where the pain arises form inflammatory bowel disease or Crohn's disease.

11. The method to treat pain according to claim-5 where the pain arises from uvetis or an inflammatory eye disease.

12. The method to treat pain according to claim-6 where the pain arises from uvetis or an inflammatory eye disease.

13. A method to treat pain arising from coronary heart disease or cardiovascular disease or peripheral artery disease by administering a metabolism blocked antifolate compound according to claim-1 with or without a pharmaceutically acceptable carrier or diluent in an amount that is therapeutically effective and nontoxic to ameliorate the said pain in a human being or a warm-blooded animal.

14. A method to treat pain arising from coronary heart disease or cardiovascular disease or peripheral artery disease by administering a metabolism blocked antifolate compound according to claim-3 with or without a pharmaceutically acceptable carrier or diluent in an amount that is therapeutically effective and nontoxic to ameliorate the said pain in a human being or a warm-blooded animal.

15. The method to treat pain according to claim-13 where the pain arises from angina pectoris or ischemia

16. The method to treat pain according to claim-14 where the pain arises from angina pectoris or ischemia.

17. The method to treat pain according to claim-13 where the pain arises from chronic obstructive pulmonary disease or asthma.

18. The method according to claim-14 where the pain arises from chronic obstructive pulmonary disease or asthma.

19. A method to treat pain arising from a solid tumor or a liquid tumor by administering a metabolism blocked antifolate compound according to claim-1 with or without a pharmaceutically acceptable carrier or diluent in an amount that is therapeutically effective and nontoxic to ameliorate the said pain in a human being or a warm-blooded animal.

20. A method to treat pain arising from a solid tumor or a liquid tumor by administering a metabolism blocked antifolate compound according to claim-3 with or without a pharmaceutically acceptable carrier or diluent in an amount that is therapeutically effective and nontoxic to ameliorate the said pain in a human being or a warm-blooded animal.

21. The method according to claim-19 where the pain arises from an epithelial tumor or a sarcoma.

22. The method according to claim-20 where the pain arises from an epithelial tumor or a sarcoma.

23. The method according to claim-19 where the pain arises from a lymphoma or leukemia or melanoma.

24. The method according to claim-20 where the pain arises from a lymphoma or leukemia or melanoma.

25. The method of treating pain according to claim-1 where the metabolism blocked antifolate compound has the L-configuration (R or S) at the asymmetric center.

26. The method of treating pain according to claim-1 where the metabolism blocked antifolate compound has the D-configuration (S or R) at the asymmetric center.

27. The method of treating pain according to claim-2 where the metabolism blocked antifolate compound has the L-configuration (R or S) at the asymmetric center.

28. The method of treating pain according to claim-2 where the metabolism blocked antifolate compound has the D-configuration (S or R) at the asymmetric center.

29. The method of treating pain according to claim-3 where the salt of the metabolism blocked antifolate compound has the L-configuration (R or S) at the asymmetric center.

30. The method of treating pain according to claim-3 where the salt of the metabolism blocked antifolate compound has the D-configuration (S or R) at the asymmetric center.
